(19) United States
(12) Patent Application Publication
(10) Pub. No.: US 2022/0387668 A1
(43) Pub. Date: Dec. 8, 2022

(54) Cultivated Autologous Limbal Epithelial Cell (CALEC) Transplantation

(71) Applicants: Massachusetts Eye and Ear Infirmary, Boston, MA (US); The Schepens Eye Research Institute, Inc., Boston, MA (US); Children`s Medical Center Corporation, Boston, MA (US); DANA FARBER CANCER INSTITUTE, INC., Boston, MA (US)

(72) Inventors: Ula Jurkunas, Winchester, MA (US); Reza Dana, Newton, MA (US); Myriam Anne Armant, Boston, MA (US); Jerome Ritz, Boston, MA (US)

(21) Appl. No.: 17/775,054

(22) PCT Filed: Nov. 9, 2020
(86) PCT No.: PCT/US2020/059687

§ 371 Date: May 6, 2022

Related U.S. Application Data
(60) Provisional application No. 62/932,473, filed on Nov. 7, 2019.

(51) Int. Cl. A61L 27/36; A61L 27/38; C12N 5/079

(52) U.S. Cl. CPC A61L 27/3604; A61L 27/3834; A61L 27/3895; A61L 27/3813; C12N 5/0621; A61L 2430/16; A61L 2430/40

(57) ABSTRACT

Provided herein are methods for generating cultivated autologous limbal epithelial cell grafts for the treatment of various disorders caused by limbal stem cell deficiency. This invention relates to methods and compositions for treating ophthalmic disorders, diseases and injuries. In particular, the field of the invention is directed to methods, kits and compositions for treating disorders, diseases, defects and injuries of the cornea and ocular surface. The present disclosure relates to preparations of cultured mammalian limbal stem cells, derived from corneal limbus tissue.

### CLAIM OF PRIORITY

This application claims the benefit of U.S. Provisional Application Ser. No. 62/932,473, filed on Nov. 7, 2019. The entire contents of the foregoing are incorporated herein by reference.

### FEDERALLY SPONSORED RESEARCH OR DEVELOPMENT

This invention was made with Government support under Grant No. #00027, “Translational Application to Manufacture Cultivated Corneal and Oral Epithelial Stem Cells for Corneal Transplantation,” Production Assistance for Cellular Therapies (PACT) awarded by the National Heart Lung and Blood Program, National Institutes of Health. The Government has certain rights in the invention.

### TECHNICAL FIELD

This invention relates to methods and compositions for treating ophthalmic disorders, diseases and injuries. In particular, the field of the invention is directed to methods, kits and compositions for treating disorders, diseases, defects and injuries of the cornea and ocular surface. The present disclosure relates to preparations of cultured mammalian limbal stem cells, derived from corneal limbus tissue.

### BACKGROUND

Corneal disease represents the second most common cause of world blindness after cataracts. The cornea is covered by a stratified squamous epithelium, which serves the dual function of protection of the ocular surface and also contributing to high quality vision. The corneal epithelium is continuous with that of the surrounding conjunctiva, the transition between the two epithelia being formed by the limbal epithelium. The basal layer of the limbal epithelium is the location for corneal epithelial stem cells, also known as limbal stem cells (LSCs).

The population of LSCs are believed to be responsible for the regenerative function allowing the maintenance of the corneal epithelium and for the barrier function against the migration of conjunctival cells onto the cornea. Significant loss of or damage to LSCs or their niche/stromal microenvironment leads to limbal stem cell deficiency (LSCD). LSCD is characterized by recurrent breakdown of the epithelium, vascularization, destruction of the corneal epithelium basement membrane, corneal scar formation, chronic inflammation and conjunctivalization of the corneal surface, which eventually leads to significant/persistent ocular pain and blindness.

While many have expanded autologous and allogeneic limbal epithelial cells in culture for generating grafts for the treatment of disorders caused by LSCD, many of the techniques have differed, not only in the expansion process, but also in the substrate to which the cells are mounted on. Further many of the techniques utilized in the art do not utilize good manufacturing practice (GMP), a practice that ensures that products are manufactured consistently with quality standards. More importantly, none of the referenced studies to date have manufactured ex vivo cultivated autologous epithelial stem cell transplants from cell suspension on denuded human amniotic membrane, without serum (human or animal), antibiotics, and xenogenic feeder cells under GMP guidelines. The use of these non-human animal-derived products in LSC expansion has several drawbacks. Firstly, such a transplant would potentially be a xenograft and, as such, the patient might require immunosuppression to prevent rejection of the tissue. Secondly, the use of non-human animal-derived products in tissue destined for human transplantation has the potential to result in interspecies pathogen transfer. This latter risk would be further augmented on a background of immunosuppression.

### SUMMARY

Provided herein are methods for preparing a cultivated autologous limbal epithelial cell (CALEC) graft for surgical transplantation, the method comprising: i) providing a tissue from a limbal biopsy or from a wet mucosal source; ii) treating the tissue from the limbal biopsy or the wet mucosal source with an enzyme blend thereby isolating limbal epithelial cells; iii) culturing the limbal epithelial cells in the presence of serum-free complete corneal epithelial cell medium for a sufficient period of time until the limbal epithelial cells reach 70-90% confluence; iv) detaching the limbal epithelial cells; v) seeding the limbal epithelial cells on a suitable membrane substrate; vi) growing the cells on the suitable membrane substrate for a sufficient period of time until the limbal epithelial cells reach 70-80% confluence thereby producing a CALEC graft; and vii) immersing the CALEC graft in a preservation medium suitable for hypothermic bio-preservation until surgical transplantation.

Also provided herein are methods of treating a limbal stem cell deficiency, comprising: i) obtaining a tissue from a limbal biopsy or from a wet mucosal source; ii) treating the tissue from the limbal biopsy or the wet mucosal source with an enzyme blend thereby isolating limbal epithelial cells; iii) culturing the limbal epithelial cells in the presence of serum-free complete corneal epithelial cell medium for a sufficient period of time until the limbal epithelial cells reach 70-90% confluence; iv) detaching the limbal epithelial cells; v) seeding the limbal epithelial cells on a suitable membrane substrate; vi) growing the cells on the suitable membrane substrate for a sufficient period of time until the limbal epithelial cells reach 70-80% confluence thereby producing CALEC graft; vii) immersing the limbal epithelial cells in a preservation medium suitable for hypothermic bio-preservation until surgical transplantation; and viii) transplanting the CALEC graft onto an affected eye of the patient.

In some embodiments, in any of the methods provided herein, the medium used in step iii) is free from non-human animal derived products.

In some embodiments, in any of the methods provided herein, the biopsy is collected in a sterile container filled with the preservation medium suitable for hypothermic bio-preservation.

In some embodiments, in any of the methods provided herein, the enzyme blend comprises collagenase class I and class II, and an animal-free serine protease.

In some embodiments, in any of the methods provided herein, the methods further comprise after step iv) washing the limbal epithelial cells with and resuspending the cells in epithelial cell culture medium.

In some embodiments, in any of the methods provided herein, the methods further comprise prior to step v) de-epithelializing the suitable membrane substrate and seeding the de-epithelialized suitable membrane substrate into a transwell insert in preparation for seeding the limbal epithelial cells.

In some embodiments, in any of the methods provided herein, the methods further comprise after step vii) maintaining the CALEC graft immersed in the preservation medium at a temperature ranging from 1-10° C.

In some embodiments, in any of the methods provided herein, step v) comprises seeding 2.5-5×10⁴ limbal epithelial cells onto the suitable membrane substrate.

In some embodiments, in any of the methods provided herein, step ii) yields 3-7×10⁴ limbal epithelial cells.

In some embodiments, in any of the methods provided herein, the sufficient period of time in step iii) ranges from 6 to 10 days.

In some embodiments, in any of the methods provided herein, the sufficient period of time in step vi) ranges from 6 to 10 days.

In some embodiments, in any of the methods provided herein, step iii) further comprises changing the medium every 2 to 3 days.

In some embodiments, in any of the methods provided herein, step vi) further comprises changing the medium every 1 to 3 days.

In some embodiments, in any of the methods provided herein, the limbal biopsy or the wet mucosal source originates from a patient suffering from a limbal stem cell deficiency.

In some embodiments, in any of the methods provided herein, the limbal biopsy or the wet mucosal source originates from an allogeneic donor. For example, in some instances, the allogeneic donor is live. Alternatively, in some instances, the allogeneic donor is cadaveric.

In some embodiments, in any of the methods provided herein, the wet musosal source is oral mucosa or conjunctiva.

In some embodiments, in any of the methods provided herein, the suitable membrane substrate is an amniotic membrane. For example, in some instances, the amniotic membrane is a human amniotic membrane. Alternatively, in some instances, the suitable membrane substrate is a basement membrane.

In some embodiments, in any of the methods provided herein, the limbal epithelial cells are positive for CD49F, CD49E, CD326, CD318, and CD340, and are negative for CD3, CD14, CD16, CD19, CD20, CD56, CD45, CD31.

Also provide herein is a population of cells produced by steps i) to iii) of any of the methods described above, wherein the cells are positive for CD49F, CD49E, CD326, CD318, and CD340, and are negative for CD3, CD14, CD16, CD19, CD20, CD56, CD45, CD31.

Provided herein also are methods of producing a population of cells positive for CD49F, CD49E, CD326, CD318, and CD340, and are negative for CD3, CD14, CD16, CD19, CD20, CD56, CD45, CD31, the method comprising: i) providing a tissue from a limbal biopsy or from a wet mucosal source; ii) treating the tissue from the limbal biopsy or the wet mucosal source with an enzyme blend thereby isolating limbal epithelial cells; and iii) culturing the limbal epithelial cells in the presence of serum-free complete corneal epithelial cell medium for a sufficient period of time until the limbal epithelial cells reach 70-90% confluence.

### Definitions

As used herein, the term “patient” or “subject” refers to members of the animal kingdom including, but not limited to, mammals, such as, human beings. The term “mammal” refers to all mammals, including, but not limited to human beings.

As used herein, the term “treatment” or “treating” a disease means administration to a patient by any suitable dosage regimen, procedure, and/or administration route of a composition, device, or structure with the object of achieving a desirable clinical/medical end-point.

As used herein, the phrase “therapeutic effect” is achieved when a desirable clinical/medical end-point has been detected.

As used herein, the phrase “diseased” or “disease-derived” or “disorder” refers to anything that is not normal. For instance, a patient (or a patient's eye) who is suffering from a limbal stem cell deficiency.

As used herein, the phrase “unaffected eye” refers to the eye in which there is no limbal stem cell deficiency. Or stated differently, the “unaffected eye” is a “healthy” eye.

As used herein, the phrase “affected eye” refers to the eye that suffers from a limbal stem cell deficiency. In some situations, both eyes are affected.

“Stem cells” are cells that exhibit self-renewal, give rise to progenitor cells, which can proliferate and differentiate to terminally differentiated cells, which are post-mitotic. For example, limbal stem cells can divide to produce limbal stem cells as well as progenitor cells. Progenitor cells can be directed to undergo differentiation (through for example culturing in vitro under appropriate condition) to, e.g., corneal epithelial cells (CECs).

“Limbal stem or progenitor cells” or “LSCs” include stem cells obtained from, e.g., the limbus, a region between cornea and conjunctiva of an eye. LSCs can proliferate and differentiate to give rise to corneal epithelial cells (CECs). In particular, LSCs are thought to reside in LSC niche within the limbus. LSCs can be isolated from limbus region comprising corneal limbus of an eye, margin between cornea and conjunctiva, border of cornea and sclera, corneoscleral limbus, a crypt region of the basal layer of limbal epithelium, a region comprising interpalisade rete ridge, or a region comprising Palisades of Vogt.

As defined herein “isolated” refers to material removed from its original environment and is thus altered “by the hand of man” from its natural state.

“Isolated limbal stem or progenitor cells” include LSCs isolated from an individual and placed in ex vivo or in vitro culture. Typically, isolated LSCs in a tissue biopsy can be dissociated to obtain single cells.

By the term “animal-free” when referring to certain compositions, growth conditions, culture media, etc. described herein, is meant that no non-human animal-derived products, such as bovine serum, proteins, lipids, carbohydrates, nucleic acids, vitamins, etc., are used in the preparation, growth, culturing, expansion, storage or formulation of the certain composition or process. By “no non-human animal-derived products/materials” is meant that the products/materials have never been in or in contact with a non-human animal body or substance.

As used herein, the term “feeder cells” is intended to mean additional cells playing a role as an aid, which are used to adjust culture conditions, for example, for target pluripotent stem cells to be proliferated or differentiated. For example, feeder cells, particularly animal feeder cells such as mouse-derived primary cultured fibroblasts, are responsible for providing a scaffold for cell adhesion and supplying growth factors required for stem cells. Accordingly, by “feeder free” or “free” of feeder cells is meant that no feeder cells are used in the preparation, growth, culturing, expansion, storage or formulation of the certain composition or process.

By the term “expanded,” in reference to cell compositions (or also referred to herein as cell populations), means that the cell population constitutes a significantly higher concentration of cells than is obtained using previous methods. For example, an “expanded” population has at least a 2 fold, and up to a 10 fold, improvement in cell numbers per gram of tissue over previous methods. The term “expanded” is meant to cover only those situations in which a person has intervened to elevate the number of the cells.

As used herein, the terms “seed” and term “passage” are well known terms in the art. They are used interchangeably herein and generally refer to a cell culture technique in which cells growing in culture that have attained a defined amount or defined confluence. The cells are removed from the vessel, diluted with fresh culture media and placed into a new tissue culture vessel to allow for their continued growth and viability.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a flow chart showing the 2-stage CALEC manufacturing process.

FIG. 2 is an image of a culture dish showing the clonal potential of limbal epithelial cells.

FIG. 3 is a graph showing the proliferation potential of limbal epithelial cells.

FIG. 4 are images of limbal epithelial cells showing their cellular distribution over time.

FIGS. 5A and 5B are images of limbal epithelial cells showing their cellular morphology over time.

FIG. 6 are images showing CALEC in situ proliferation and viability.

FIG. 7 is an image of cells to illustrate the cell-counting method.

FIG. 8 are images of cells showing how dead cells are counted by fluorescence microscopy.

FIG. 9 is a graph showing correlation between LDH release and % dead cells in CALEC constructs.

FIG. 10 is a graph showing recovery of metabolic activity after hypothermic storage.

FIG. 11A is a graph showing LDH levels in supernatants and FIG. 11B are images of cells showing live/dead cells by fluorescence microscopy.

FIG. 12 is a schematic showing the three conditions tested to measure the impact of hypothermic biopreservation on CALEC stability.

FIG. 13 is a graph showing CALEC lactate dehydrogenase (LDH) release.

FIG. 14 is a graph showing CALEC glucose consumption and lactate production.

FIG. 15 is a schematic showing surgical transplantation of CALEC graft.

FIG. 16 is a schematic demonstrating CALEC clinical manufacturing.

FIGS. 17A-17F are images showing the evaluation of the ‘sternness’ in CALEC construct.

### DETAILED DESCRIPTION

There remains a need for an improved ex vivo LSC expansion method that provides cells suitable for direct transplantation into a patient and which does not suffer from the disadvantages of the prior art.

The methods described herein include many advantages over the prior art. Specifically, unlike methods described in the prior art, the manufacturing procedures described herein do not require animal serum, xenogenic (murine) feeder cells, antibiotics, and the methods herein use only GMP-grade materials. The methods described herein rely entirely on well-defined reagents at all steps in the manufacturing process. Further, the methods described herein would not require immunosuppression to prevent rejection since the stem cells are autologous.

### Methods of Preparing Limbal Stem Cells for Surgical Transplantation

### Limbal Biopsy:

Limbal biopsy can be obtained from the tissue of an unaffected/healthy eye of a patient suffering from limbal stem cell deficiency by any suitable and standard means that are known in the art. In some instances, the tissue from the limbal biopsy can originate from the patient suffering from the limbal stem cell deficiency or can originate from an allogeneic donor. For example, the limbal biopsy can be obtained from the tissue of an unaffected/healthy eye of a live allogeneic donor. Alternatively, the limbal biopsy can be obtained from the tissue of an unaffected/healthy eye of a cadaveric allogeneic donor. The tissue from the limbal biopsy is stored in a container filled with a preservation medium suitable for hypothermic bio-preservation, such as HypoThermosol® FRS available from Sigma-Aldrich.

### Wet Mucosa Biopsy:

In some diseased states, like aniridia and Stevens-Johnson syndrome, both eyes are affected (i.e., diseased) and as such a biopsy cannot be obtained from a healthy or unaffected eye. In these situations, a biopsy can be obtained from the oral mucosa, conjunctiva, or any other suitable wet mucosal tissue. Stem cells from the wet mucosal tissue can be cultured in a similar manner and used for corneal transplantation. Since they are autologous, the patients would not require immunosuppression and bilateral cases can be treated.

### Enzymatic Digestion

Limbal epithelial sheets can be isolated from the biopsy by enzymatic digestion. An exemplary protocol is as follows. First, the biopsy is rinsed in a saline solution, such as PBS or a suitable equivalent, and then transferred into an enzyme blend of highly purified collagenase class I and class II, which are blended in a precise ratio with each other and with a medium concentration of highly purified thermolysin. A commercially available suitable enzyme blend is Liberase MNP-S from Roche. The biopsy is incubated in this enzyme blend for 30±5 minutes at 37° C., 5% CO₂. At the end of incubation the biopsy is transferred into a solution of animal-free serine protease (such as TrypLE™ Select CTS™ available through ThermoFisher Scientific), where epithelial sheets are peeled off and dissociated into a single cell suspension. After enzymatic digestion of the biopsy, the cells are centrifuged (450 G) for 5 minutes at room temperature. The supernatant is collected for sterility testing (e.g., BacT, see below). The cells are resuspended and plated into 1 well of a 6-well culture plate, in serum-free complete corneal epithelial cell media (such as the one available through ATCC) and placed in a humidified incubator at 37° C., 5% CO₂.

### Culturing the Limbal Epithelial Cells

The isolated limbal epithelial cells can be cultured in the presence of serum-free complete corneal epithelial cell media (such as the one available through ATCC). Cell growth is monitored under the microscope and media is changed every 2-3 days until culture reaches 70-90% confluence, which takes approximately 6-10 days.

### Detaching the Limbal Epithelial Cells

At the end of the prior step (primary culture (P0)), cells are detached from the substrate, e.g., by trypsinization using an animal-free serine protease (such as TrypLE™ Select CTS™ available through ThermoFisher Scientific). The cells can be subsequently harvested by standard methods and centrifuged (e.g., 450 G, 5 minutes at room temperature). The supernatant is collected and archived. The cells can then be resuspended in serum-free complete corneal epithelial cell media (such as the one available through ATCC).

### Seeding and Growing the Limbal Epithelial Cells on a Suitable Membrane Substrate

The limbal epithelial cells are then seeded on a suitable membrane substrate. The membrane substrate may be an aminiotic membrane, such as a human amniotic membrane. A suitable commercially available one is AmnioGraft® from BioTissue. Another suitable membrane substrate is a basement membrane substrate. Basement membranes provide an adhesive substrate for cells, and they are linked functionally to the actin cytoskeleton via integrins or other ECM receptors to mediate cell attachment and migration, as well as modulating intracellular signaling pathways.

Prior to seeding the limbal epithelial cells on a suitable membrane, the membrane can be de-epithelialized by standard means and immobilized inside a transwell insert. The transwell insert can be placed inside a single well of a 6-well plate containing complete serum-free corneal epithelial cell growth media (such as the one available through ATCC). A sufficient number of cells, e.g, approximately 2.5-5×10⁴ cells, can then be seeded onto the membrane. One media change can be done around 48 hours after the seeding followed by daily media change until 90-100% confluency is reached. Cell growth can be monitored under the microscope until culture reaches confluence (P1). A sterility test (e.g., BacT) can be performed on the used media at 48 h before end of culture.

### Markers

The methods provided herein the yields epithelial cells with a consistent phenotype and this identity is maintained throughout the manufacturing process. Specifically, cells isolated after enzymatic digestion, at the end of primary culture (P0), and cells that are on the final CALEC product, maintain a consistent phenotype. P0 cells are positive for epithelial cell markers (CD49F, CD49E, CD326, CD318, CD340), negative for hematopoietic lineage-specific markers (CD3, CD14, CD16, CD19, CD20, CD56 and pan-leukocyte marker CD45) as well as endothelial marker (CD31). These cells express CD44 and CD73 but unlike mesenchymal stem cells, limbal cells do not express CD105 or CD13. Cells at the end of the manufacturing process have the same phenotype.

### Storage Until Surgical Transplantation

When confluent, the cells can be counted with a microscope (target: 0.4 to 1×10⁶ cells). The cellular graft can be rinsed, e.g., with 0.9% sodium chloride and then transferred to a hypothermic bio-preservation solution (e.g., HypoThermosol® FRS, Bio Life Solutions) and maintained for transport and storage, e.g., at 1-10° C. in a container, until transplantation, which preferably should occur within 24 hours to 48 hours, more preferably within 24 hours. The graft product(s) can be hand-carried, e.g., in an appropriate transport container(s), to the participant location by trained staff members or approved courier. Final culture supernatant can be used for quality control testing (e.g., viability (e.g., LDH assay), sterility (BacT), mycoplasma, endotoxin, and gram staining.

### Quality Control

Various quality control testing can be done at any time throughout the methods described herein. Quality control (QC) testing pre- and post-release are summarized below in Tables 1 and 2. Table 1 represents exemplary requirements for product release (acceptance criteria). Table 2 represents exemplary criteria for post-release.

Viable cell count can be obtained using standard methods known in the art, such as the Trypan Blue exclusion method.

Sterility is defined as the complete absence of viable microorganisms capable of developing and multiplying under favorable conditions. According to the pharmacopoeia compendia, the sterility of pharmaceutical products should be confirmed by subjecting a representative sample of the product to sterility testing; the product is considered to be non-sterile when microbial growth is detected during the test (Baird, 2004; Denyer and Baird, 2007; Pinto et al., 2010; Sandie, 2012).

Sterility testing can be done by any suitable means. For instance, in some embodiments, sterility testing can be performed using the bioMérietvc's BacT/Alert® 3D Microbial Detection System. The BacT/Alert system was found to have a quicker time to detection at the same sensitivity level. Samples are directly inoculated into the BacT culture bottles (AST and NST). The bottles are then loaded into the BacT/Alert® 3D analyzer, and incubated for 14-days at 35° C. If microorganisms are present in bottles, they will produce carbon dioxide as they metabolize substrates in the media. This carbon dioxide changes sensors at the bottom of the bottles from blue-green to yellow. Using this sensor and reflected light, the BacT/Alert monitors the production of carbon dioxide and signals users if it determines bottles to be positive. If, at the end of 14-day incubation, the system does not determine bottles to be positive, it will report them as negative. If the system detects CO₂ production it will report that bottle as positive. If a positive is detected during the 14-day incubation phase, the bottle will be off loaded and a sample sent for identification and sensitivities.

Viability is measured indirectly by measuring lactate dehydrogenase (LDH) activity released from the cytosol of damaged cells into the supernatant. For instance, this can be done by using a colorimetric assay for the quantification of lactate dehydrogenase (LDH) activity released from the cytosol of damaged cells into the supernatant.

To verify the identity of isolated limbal epithelial cells, any suitable means known in the art can be employed. For instance, a standard FACS assay with pre-validated markers can be conducted prior to seeding the membrane. A suitable panel of markers include the following antibodies: CD45, CD31, CD13, CD49F, CD340 and a viable cell dye.

To ensure morphology of the isolated limbal epithelial cells, the cells can be observed using a microscope, such as an inverted microscope. The cells should form a continuous layer of polygonal/cuboidal epithelial cells, covering the area within the O-ring.

A colony forming efficiency (CFE) assay can be performed as a functional measurement of limbal progenitor cells. Cells at P0 are plated at low density (1-2,00 cells/cm²) and placed in culture (37° C., 5% CO2, 90% RH). After 10-14 days, colonies are fixed and stained with Rhodamine B. Colonies are scored under an inverted scope and the CFE (%) is expressed as the ratio of the number of colonies counted to the number of inoculated cells and multiplied by 100(%). This assay can serve as part of a potency assay for the initial phase of the clinical trial.

For the proliferation potential assay, cells at P0 can be plated at 3 different concentrations (125, 250 and 500 cells/well) with 6 replicates/concentration in a 96 well plate in corneal growth media at 37° C., 5% CO₂, 90% RH. After 7 days, proliferation can be measured by enumerating the mean intracellular ATP (iATP)/well on a luminometer. A reference ATP standard and controls are included in the assay. This assay can be performed as part of a potency assay for the isolated limbal epithelial cells.

Mycoplasma testing can be done through any suitable means known in the art, such as through the use of a Polymerase Chain Reaction kit with primers specific for mycoplasma sequences.

Endotoxin levels can be determined by suitable means, such as the gel-clot Limulus Amebocyte Lysate (LAL) test method. This method uses a small volume of the final culture supernatant, collected from isolated limbal epithelial cells, mixed with an equal amount of the Limulus Amebocyte Lysate. If endotoxins are present in the sample they will be observed by seeing a clot formation. (The sensitivity of this assay is 0.06 EU/mL.) If the result is below 0.5 EU/mL, it will report that the isolated limbal epithelial cells can be used for the transplant.

Gram staining methods are known in the art. The preferred result is “No Organism Seen.”

TABLE 1
Quality Control tests for limbal epithelial cell product

| | | Exemplary | Acceptance |
|---|---|---|---|
| | | Method to be | Criteria for |
| Processing step | Test | Employed | Release |
| Initial Biopsy at Day | Sterility | BacT/Alert | No growth at release |
| 0 | | | |
| Post Wash (Post | Sterility | BacT/Alert | No growth at release |
| enzymatic digestion, | | | |
| pre-culture) | | | |
| End of primary culture | Viable | Trypan Blue | 3-5 × 10⁴ cells per |
| (P0) | Cell counts | exclusion | Amniograft |
| | for seeding | | |
| | Sterility | BacT/Alert | No growth at release |
| Supernatant collected | Sterility | BacT/Alert | No growth at release |
| 24-48-h before end of | | | |
| culture (P1) | | | |
| Final culture | Viability | LDH assay | ≤150 U/L |
| supernatant collected | | | |
| | Endotoxin | LAL | ≤0.5 EU/ml |
| | Gram-Stain | Gram stain | No organism seen |
| | Sterility | BacT/Alert | Sample Taken |
| CALEC Final product | Cell count | Microscopy | ≥0.4 × 10⁶ cells per |
| | | (EVOS ®) | Amniograft |
| | morphology | Microscopy | Continuous layer of |
| | | (EVOS ®) | polygonal epithelial |
| | | | cells |
| | Pre-infusion | PCR | Sample Taken |
| | Final | | |
| | Supernatant | | |

TABLE 2
Post-release Quality Control tests for CALEC product

| Processing | | | |
|---|---|---|---|
| step | Test | Method | Acceptable Criteria |
| Post- | Sterility of Initial | BacT/Alert | No growth within 14 |
| Release | Biopsy at Day 0 | | days of inoculation |
| | Sterility of End of | BacT/Alert | No growth within 14 |
| | primary culture (P0) | | days of inoculation |
| | Sterility of Supernatant | BacT/Alert | No growth within 14 |
| | collected 24/48 hours | | days of inoculation |
| | before end of culture | | |
| | (P1) | | |
| | Sterility of Final | BacT/Alert | No growth within 14 |
| | culture supernatant | | days of inoculation |
| | Mycoplasma Detection | PCR | No Mycoplasma |
| | of Pre-infusion | | Detected |
| | Final Supernatant | | |

### Methods of Treating Limbal Stem Cell Deficiency in a Patient

Provided herein are methods of treating limbal stem cell deficiency in a subject, e.g., a mammalian subject, e.g., a human or non-human veterinary subject. The limbal epithelial cell graft product produced by the methods described above can be transplanted into a subject, e.g., a subject in need thereof. In some embodiments, the subject with a limbal stem cell deficiency has symptoms including blurry vision, a foreign-body sensation, photophobia, tearing, and/or pain. A treatment as described herein can result in an improvement in any one of more of those symptoms, and/or a return to normal vision for that subject.

For each product, an estimate of the cell dose can be determined in situ by scanning the surface of limbal epithelial cells within the O-ring with an imaging system (such as the EVOS® cell imaging system, Life Technologies/Thermo-Fischer). A number of areas, e.g., three to six, e.g., five areas, of the scan can be counted and averaged. The average count/μm² can then be used to calculate a cell dose by multiplying by the surface area of the trephined piece. For most subjects, the transplanted surface will be 1.5 cm².

Standard methods and standard care are employed during the transplantation. For instance, prior to biopsy and/or transplantation, suitable antibiotics and/or anesthetics can be administered. In cases where the subject has one affected and one unaffected eye, both the biopsied (unaffected) eye and operative (affected) eye can be cleaned. The surgery is performed under sterile conditions. Suitable antibiotics and/or pain medications can be employed during or post-surgery.

### Kits for Treating Limbal Stem Cell Deficiency in a Patient

Additionally provided herein are kits that include the necessary reagents needed to accomplish the methods described herein. For instance, provided herein is a kit including a container with a preservation medium suitable for hypothermic bio-preservation for storing a limbal biopsy, an enzyme blend, serum-free complete corneal epithelial cell medium, a suitable membrane substrate, and any other suitable reagents and tools for culturing limbal epithelial cells and for transportation of a CALEC graft.

### EXAMPLES

The invention is further described in the following examples, which do not limit the scope of the invention described in the claims.

### Example 1: Generation of a CALEC Graft

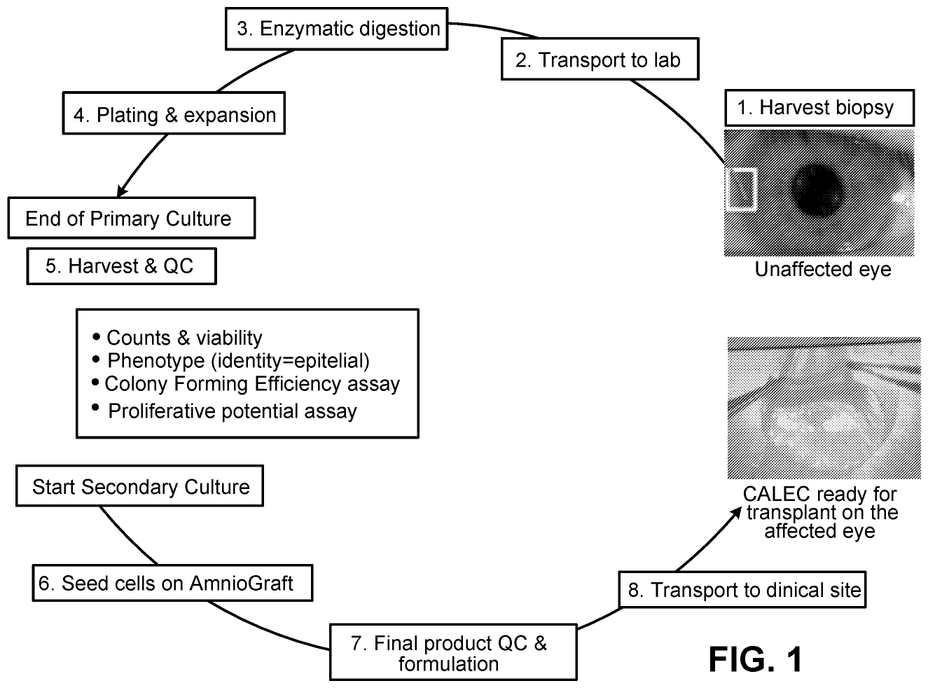

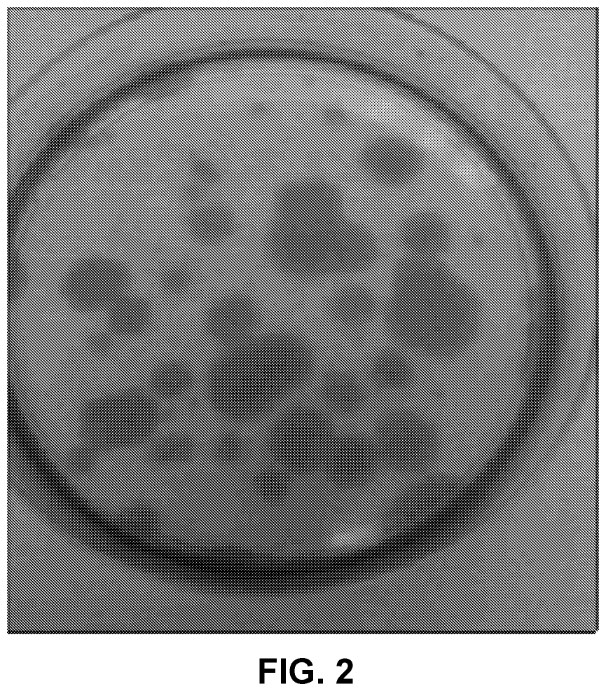

Existing methods and reagents were improved upon to reproducibly and safely yield a transplantable cellular graft within 3 weeks. A 2-stage manufacturing process was validated (FIG. 1) where enzymatically-isolated limbal epithelial cells (primary culture) were first expanded. Then a fraction of the expanded cells (P0) were seeded onto a transplantable substrate, an amniotic membrane (AmnioGraft®) for further expansion (secondary culture).

To develop this robust manufacturing process, starting materials and supplies were carefully selected and qualified. We identified serum-free epithelial cell culture media, GMP-grade enzyme and an FDA-approved membrane as a transplantable scaffold (AmnioGraft®). Every step of the manufacturing process was optimized and standardized to enhance the quality and consistency of the manufactured tissue. In-process quality control testing and final product characterization were also optimized and standardized. CALEC grafts pose distinct challenges, as the final transplantable product cannot be directly sampled for testing. However, during process development we used multiple “destructive” methods to assess the quality of CALEC grafts in terms of cellular composition, cell distribution and morphology, cell viability and cell proliferation.

### In Process Characterization of CALEC Manufacturing.

Flow cytometry assays: To develop the manufacturing procedure, biopsies from cadaveric corneas (National Disease Research Interchange, PA) were processed for limbal epithelial cell isolation. The average viable cell yield per biopsy post-isolation was 5±2×10⁴ cells (n=105). From these cells, only a fraction actually generated colonies once they were seeded into culture and progressively covered the well. At the end of the primary culture, cells were harvested, counted and the cellular composition was characterized by flow cytometry. On average, the viable cell count at P0 was 3.8±2.1×10⁵ cells. This was sufficient for downstream processing, QC testing and seeding of the AmnioGraft®. Extensive phenotypic characterization by flow cytometry demonstrated P0 cells were positive for epithelial cell markers (CD49F, CD49E, CD326, CD318, CD340), negative for hematopoietic lineage-specific markers (CD3, CD14, CD16, CD19, CD20, CD56 and pan-leukocyte marker CD45) as well as endothelial marker (CD31). These cells express CD44 and CD73 but unlike mesenchymal stem cells, limbal cells do not express CD105 or CD13. From these initial studies we established a basic 6-color panel (5 markers and viability) to monitor the identity of CALEC products at different stages of the processing.

| | Lineage-specific marker | | | | |
|---|---|---|---|---|---|
| | Hemato- | Endo- | | | |
| | poietic | thelial | Mesenchymal | Epithelial | |
| Markers | CD45 | CD31 | CD13 | CD49F | CD340 |
| Expression | − | − | − | + | + |

Using this panel we examined the cellular composition immediately post enzymatic isolation of limbal cells from the biopsy, at the end of primary culture (P0) and the final CALEC product (cells were recovered from the membrane by enzymatic digestion with TrypLE™ Select). These studies confirmed that the limbal cell isolation method yields epithelial cells with a consistent phenotype and this identity is maintained throughout the manufacturing process. P0 cells are representative of cells on the AmnioGraft® and this panel is included as a QC test at P0.

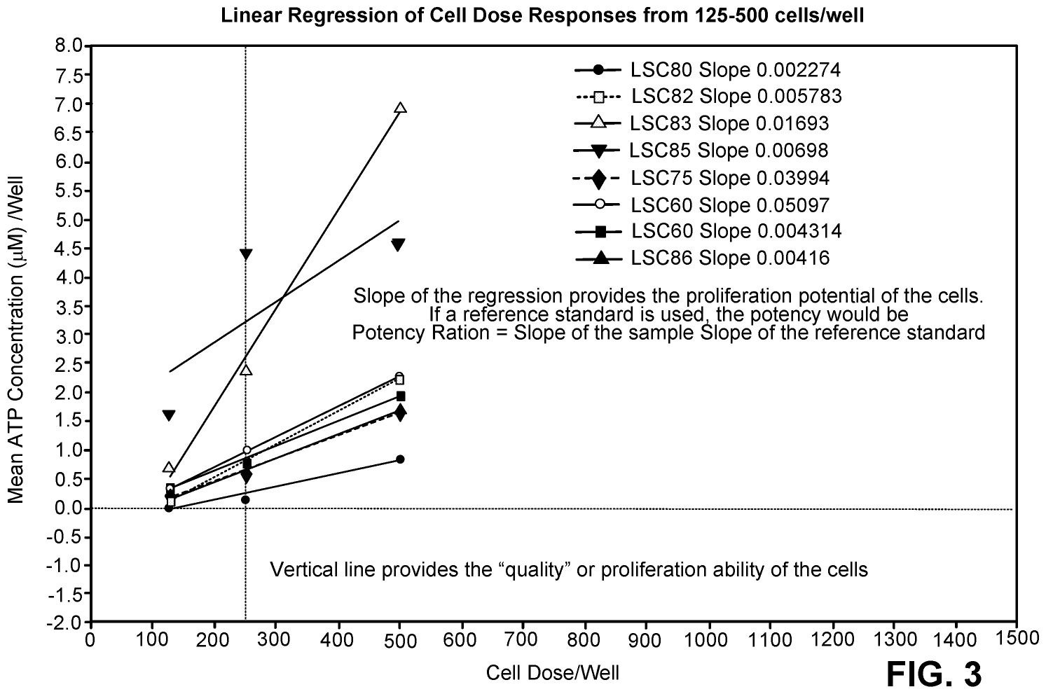

Proliferation potential assay: We also developed a quantitative assay to evaluate the proliferative potential of limbal epithelial cells by measuring intracellular ATP (iATP). Changes in iATP concentration as a result of mitochondrial activity correlate directly with cell number and proliferation. Cells are harvested at the end of the primary culture (P0). Three different cell doses (125, 250 and 500 cells/well) were plated in 6 replicates in a 96 well plate in corneal growth media and placed at 37° C., 5% CO₂, 90% RH. iATP concentration was measured after 7 days by bioluminescence (Tecan Genios). ATP standards, controls and enumeration reagents were all from Hemogenix®. The results obtained in 8 independent experiments were shown in FIG. 3. The slope of the linear regression provides the proliferation potential of each sample. In FIG. 3, sample LSC83 has a higher proliferation potential than other samples in the group. Whether results from this assay correlate with in vivo outcome will be evaluated in the clinical trial.

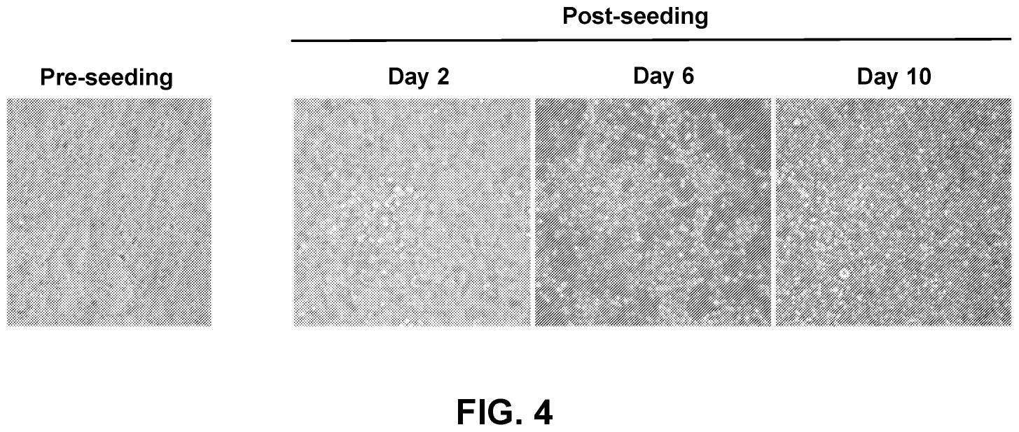

Secondary culture—seeding of the AmnioGraft®: When limbal epithelial cells at P0 (4±1×10⁴ cells) were seeded onto the denuded AmnioGraft®, they grew and progressively covered the entire surface available as depicted in FIG. 4. This took a mean of 8.8±2.5 days and was monitored with an inverted microscope. An entire scan of the area was performed to confirm uniform coverage within the O-ring.

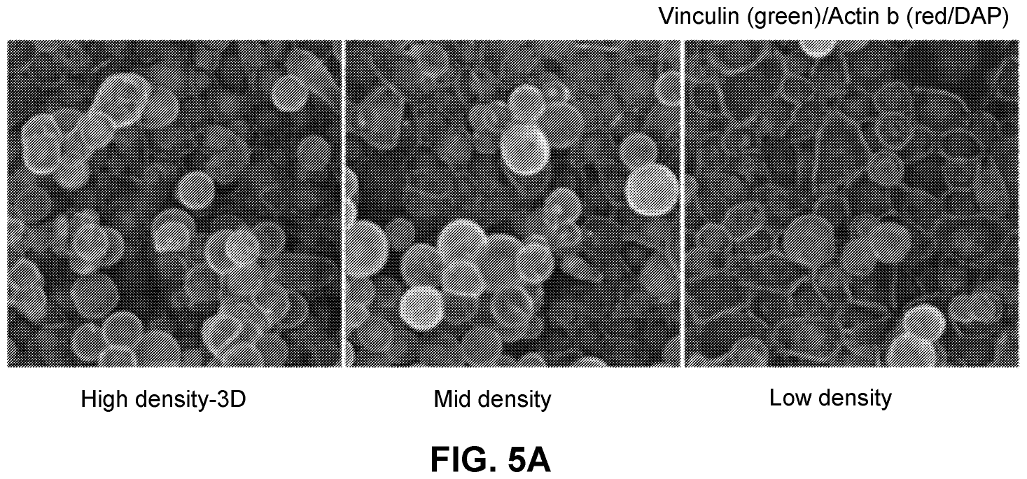

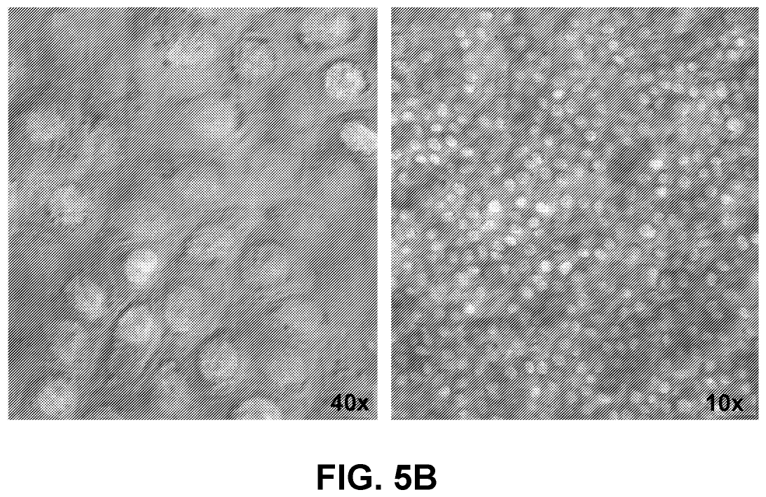

To determine the optimal cell number for seeding the amniotic membrane, we used confocal microscopy to assess the quality of the surface coverage of the final product. We found that the number of cells used at seeding as well as time in culture were important parameters that define CALEC morphology (FIG. 5A). To standardize the process and obtain a reproducible cellular construct, the ideal cell number to seed the amniotic membrane is set at 4±1×10⁴ cells. The minimum acceptable number is 2.5×10⁴ cells. This results in a uniform layer of cobblestone shaped cells as shown in FIG. 5B.

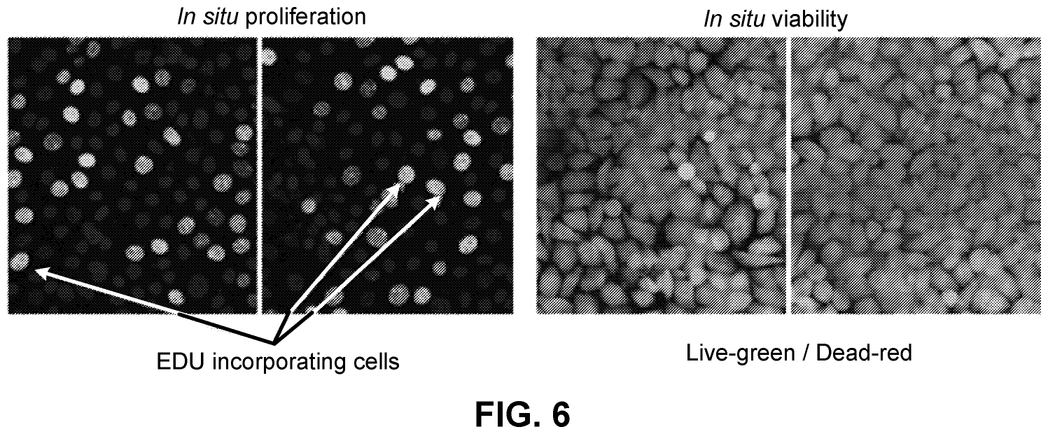

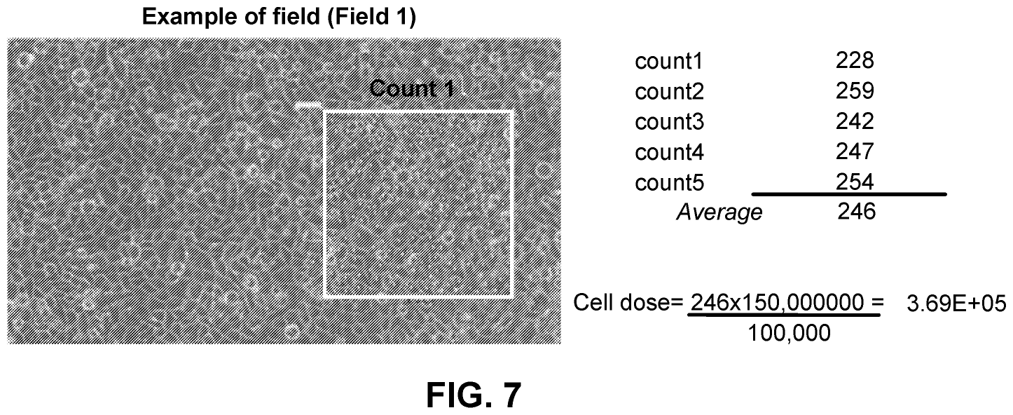

Other CALEC in situ quality assessments: To assess the quality of the final product we also evaluated in situ proliferation and viability at the end of the process (before formulation for transport). To evaluate proliferation, CALEC constructs were pulsed with the modified thymidine analog, 5-ethynyl-2′-dexyuridine (EdU) for 4 hrs and then processed for analysis (left panels FIG. 6). EdU was efficiently incorporated into newly synthesized DNA and fluorescently labeled with a bright, photostable Alexa Fluor® dye. Nuclei were counterstained with DAPI and images were acquired on an Olympus FluoView™ FV1000 confocal microscope. To assess viability, constructs were stained with LIVE/DEAD® assay (Life Technologies) and images were immediately acquired on an Olympus FluoView™ FV1000 confocal microscope (right panels FIG. 6). Shown are 2 independent fields of the same construct. Cells that are synthesizing new DNA and incorporating EdU are green/light blue while EdU negative cells are dark blue. The EdU+ and EdU− cells can be counted and the percentage of proliferating cells can be estimated manually or using automated image analysis software (Imaris, Bitplane). An average of 25±2% of cells were estimated as synthesizing new DNA. Similarly, the in situ viability assay shows that 17±1% of cells are not viable. These studies demonstrate that the cellular grafts obtained following our manufacturing method are healthy and dynamic structures.

Cell counting method for the final CALEC product: For each product, an estimate of the cell dose in each construct was determined in situ by scanning the surface of CALEC within the O-ring with an imaging system (EVOS® cell imaging system, Life Technologies/Thermo-Fischer). 5 independent fields were photographed. On each field, cells were counted manually within a 0.1 mm² (100,000 μm²) area and the average of the 5 counts was calculated. The average count/μm² was used to calculate the cell dose in the final CALEC construct by multiplying by the surface area of the trephined piece. For most patients, the transplanted surface will be 1.5 cm² (150,000,000 μm²).

CALEC surrogate viability assay: Lactate dehydrogenase (LDH) is a well-established marker/indicator of cellular toxicity and lysis. LDH is a cytosolic enzyme that is released when the plasma membrane is damaged. Previous studies have established a good correlation between the quantity of LDH released by cells in the culture supernatant and the percentage of dead cells counted by Trypan Blue staining. To validate the LDH assay for monitoring cultures of limbal epithelial cells we collected supernatants to measure LDH release and also determined % dead cells (and live cells) using a different method. We opted for the LIVE/DEAD® viability assay which is a two-color assay to determine viability of cells in a population based on plasma membrane integrity and esterase activity. This method discriminates live from dead cells by simultaneously staining with green-fluorescent calcein-AM to indicate intracellular esterase activity and red-fluorescent ethidium homodimer-1 to indicate loss of plasma membrane integrity.

Limbal epithelial cells were isolated and expanded from 4 independent biopsies. P0 cells were harvested and seeded onto (7) 35 mm dishes (2×10⁴/dish) in corneal growth media for 5 days. Culture supernatants from the 7 dishes were collected and pooled; this is the 0 h time-point QC sample (or final culture supernatant) for LDH release assay. 1 dish was immediately processed for LIVE/DEAD® staining at 0 h and the other 6 dishes were placed in HypoThermosol® FRS and transferred to hypothermic conditions (2-8° C.). Every 24 h, a dish was taken out of the cold, HypoThermosol® FRS was replaced with corneal growth media and the dish was put in the culture incubator (37° C., 5% CO2, 90% RH) for 24 hrs before culture supernatant was collected for LDH assay (24 h, 48 h, 72 h, 96 h, 120 h and 144 h time points) and cells were processed for LIVE/DEAD® staining. Cell culture supernatants were spun down and cell-free supernatants were stored at −30° C. until the LDH assay was performed.

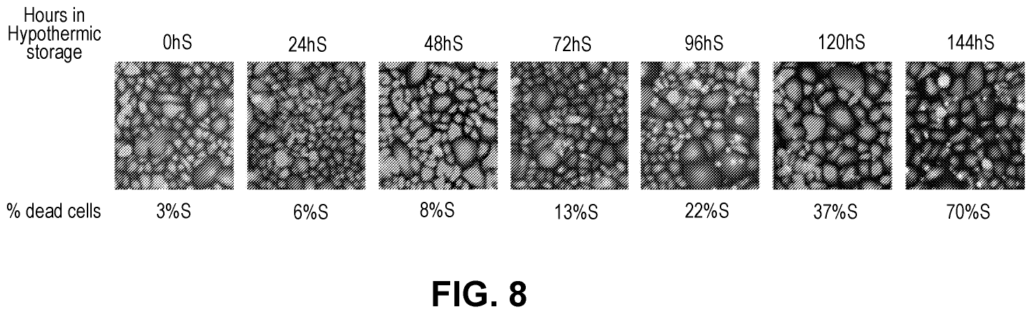

Immediately after the supernatant was removed, cells were stained with a combination of calcein-AM (green fluorescence labeling live cells) and ethidium homodimer-1 (red fluorescence labeling dead cells) and analyzed under a fluorescence microscope (Floid® cell imaging station). For each dish, 5 different fields were captured and for each field the % of dead cells was calculated as follow: % Dead Cells=(#dead cells)/(#dead cells+#live cells)*100. An example of 1 field for each time point is shown in FIG. 8.

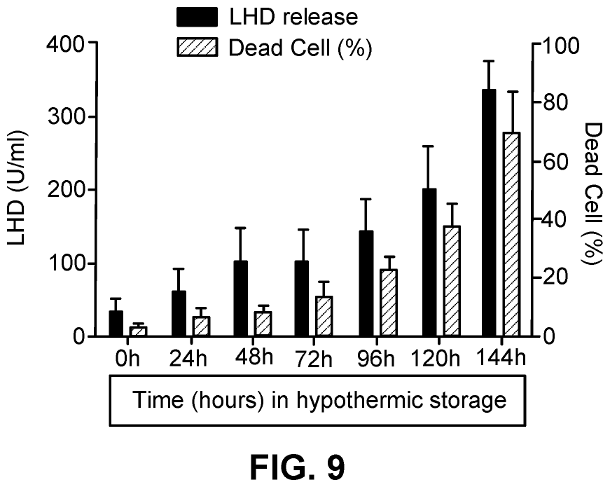

LDH was measured by a commercial colorimetric assay (Roche Diagnostics) on the CEDEX bio analyzer (Roche Diagnostics). The average LDH value±standard deviation of the 4 experiments was shown in FIG. 9. This shows that the amount of LDH released in the culture supernatant increases over time and parallels the progressive increase in the percentage of dead cells as determined by LIVE/DEAD® stain. The calculated correlation coefficient is r=0.99.

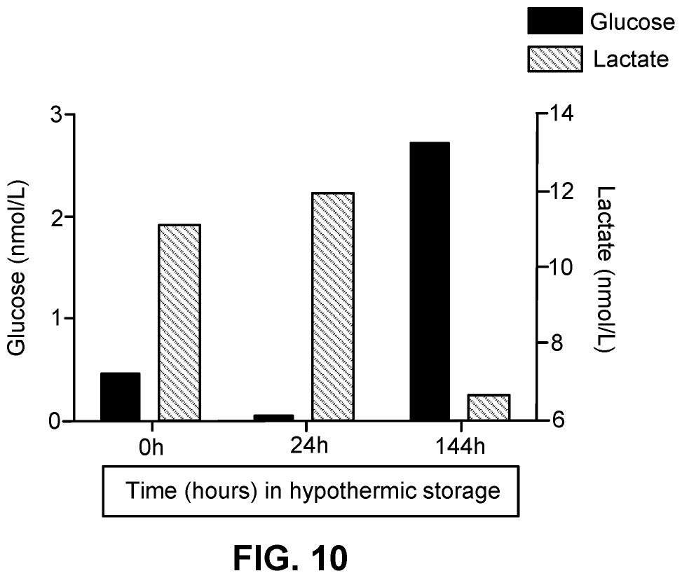

In addition to measuring viability, we examined cell metabolic activity measured by glucose consumption and lactate production (CEDEX Bio analyzer, Roche Diagnostics) pre (0 h) and post (24 h, 144 h) hypothermic storage. We used the same supernatants where LDH was measured. Results from a representative experiment are shown in FIG. 10. These results show that when cells have spent <24 h in hypothermic storage, they remain healthy (LDH<100 U/ml and <10% dead cells) and metabolically active as shown by their ability to consume glucose and produce lactate. However, when cells have been kept in hypothermic storage for 144 h the LDH levels increased (>250 U/ml) and the % dead cells is >50% indicating that the cells cannot recover their metabolic activity.

The functional assessment of metabolic activity is very sensitive and complements the LDH assay. There is a quantitative correlation between the LDH released in the medium and the percentage of dead cells. We therefor conclude that the LDH assay can be used to monitor cell death and viability of the CALEC product in our manufacturing process. Based on these results, we have established the acceptance criteria for LDH value measured in the final CALEC supernatant at <150 U/ml, which in our correlation studies represents approximately 70% viability. In repeated measurements during process development, LDH values in final CALEC supernatant routinely correspond to a viability >90%.

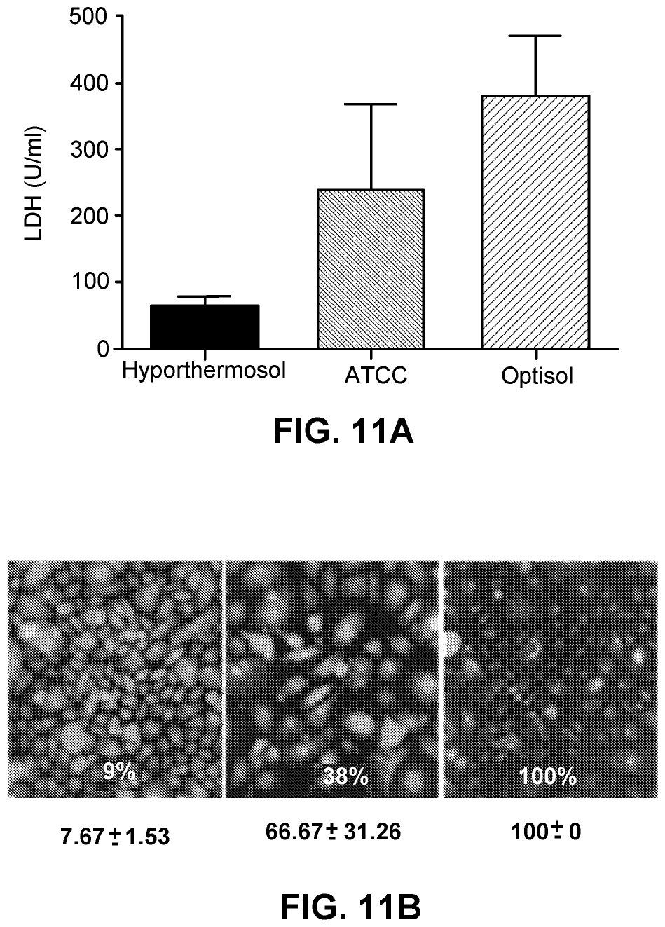

CALEC formulation for transport: Identifying the optimal final formulation for storage and transport of CALEC from the manufacturing facility to the hospital operating room was an important step in process development. To address this issue, we plated limbal epithelial cells at P0 and stored them at 4° C. in 3 different media: HypoThermosol® FRS, ATCC corneal epithelial cell media and Optisol (cadaveric cornea storage and transport specialized media). After 24 hours, dishes were removed from cold, the storage media was replaced with fresh corneal media (ATCC) and returned to incubator (37° C., 5% CO₂, 90% RH) for 24 hours before LDH was measured in the supernatants (FIG. 11A) and cells were stained with LIVE/DEAD® (FIG. 11B).

Limbal epithelial cells maintained in hypothermic storage in HypoThermosol® FRS had the lowest LDH value (67.46±11.41, n=3) and lowest % dead cells (7.67±1.54, n=3). Cold storage in either ATCC or Optisol resulted in lower viability.

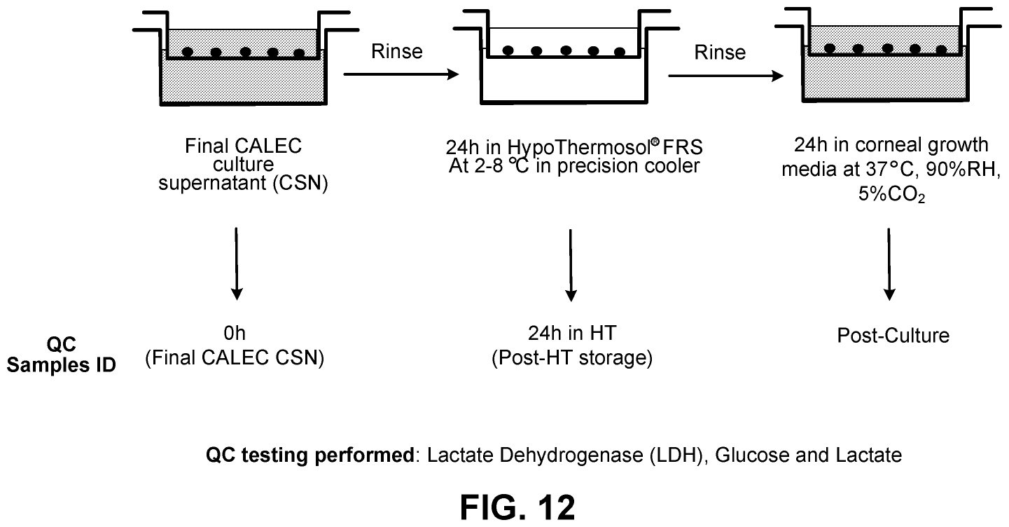

Based on experimental data with P0 cells shown in FIG. 11, we selected HypoThermosol® FRS as CALEC final formulation media and transport at 2-8° C. Further experiments were performed to confirm the results on actual CALEC constructs. To measure the impact of hypothermic biopreservation on CALEC stability these additional experiments were performed using conditions that better mimic clinical storage and transport conditions shown in FIG. 12. 8 independent CALEC grafts were generated following standardized procedures. At the end of manufacturing, the final CALEC culture supernatant (CSN) was collected for quality control and CALEC was rinsed twice and transferred in HypoThermosol® FRS and placed in the precision thermal cooler. After 24 hours in hypothermic storage, the product was taken out of the cooler, and the hypothermic solution was removed and kept for quality control (24 h in HT, post-HT storage). CALEC was rinsed twice with a clinical grade saline solution (BSS®, Sterile Irrigation Solution, that will be used by the surgeon) and then placed back into complete corneal growth media and put in the incubator (37° C., 5% CO2, 90% RH) for 24 hrs. At the end of the culture period, the supernatant (Post-Culture) was also collected for QC. The 3 QC samples were analyzed for LDH release and metabolic activity (Glucose consumption and Lactate production) using the CEDEX bio-analyzer (Roche Diagnostics).

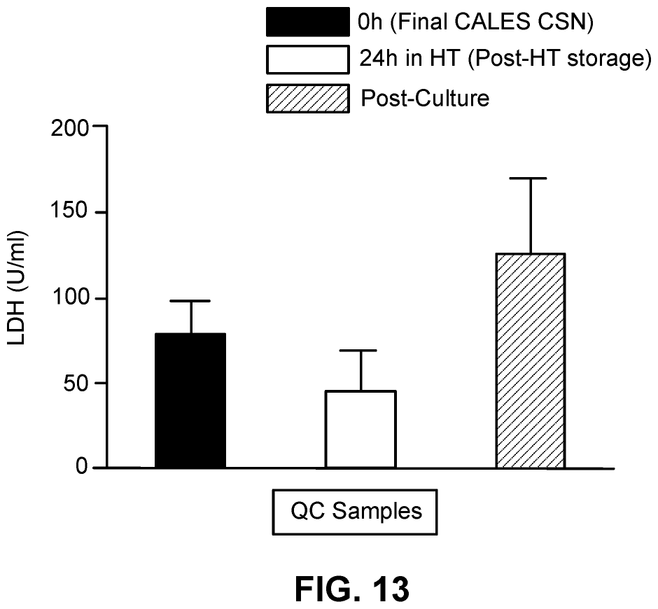

FIG. 13 shows the results of LDH release by CALEC in the final supernatant, after 24 h in HypoThermosol® FRS and post-culture recovery (average ±standard deviation, n=8). These LDH values correspond to <20% dead cells and >80% viability.

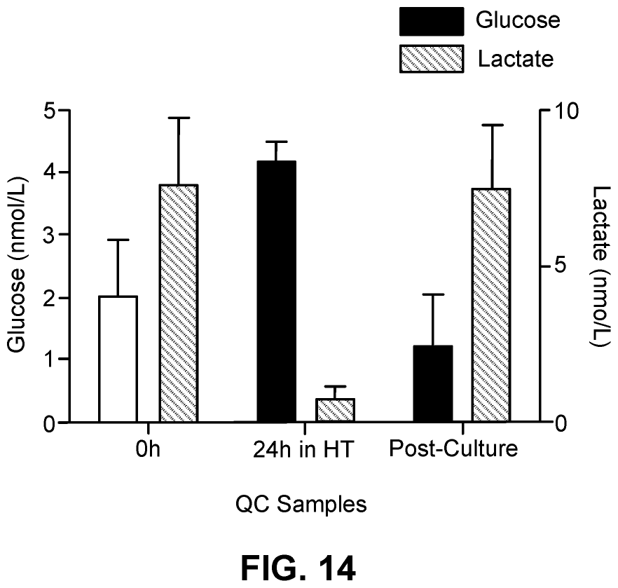

An excellent biomarker for stability is the ability of the cellular graft to recover full metabolic activity (as measured by glucose consumption and lactate production) when put back in culture. Based on our experience, this is an appropriate functional approach to examine the impact of hypothermic storage on the graft. This is shown in FIG. 14 (average±standard deviation, n=8).

The corneal media and HypoThermosol® FRS contain approximately 5 nmol/L and 4 nmol/L of glucose (and no lactate) respectively. Analysis of the final supernatant (0 h) shows that CALEC is metabolically active. As expected from our initial studies aimed at identifying a suitable hypothermic biopreservation solution, CALEC placed in HypoThermosol® FRS for 24 h at temperatures ranging from 2-8° C. are quiet metabolically (very low glucose consumption and lactate production). When placed back at 37° C., 5% CO2, 90% RH in corneal culture media for 24 hrs, limbal epithelial cells on the graft completely recovered their metabolic activity, consuming glucose and producing lactate. These results demonstrate that CALEC constructs are stable when placed in HypoThermosol® FRS for 24 h in the same storage and transport cooler that will be used during the clinical trial. When transferred to HypoThermosol® FRS and hypothermic storage conditions, the graft becomes metabolically quiescent. This is expected and desirable. Most importantly, once the CALEC graft is returned to growth conditions, the cells completely recover their metabolic activity.

Two important metrics summarize the feasibility and reproducibility of CALEC manufacturing from our in vitro limbal biopsy model.
Cellular yield at P0: the average cellular yield at P0 is 3.8±2.1×105 cells. This is sufficient to seed AmnioGraft® and perform QC testing. Of note, no cells could be grown out of central cornea or conjunctiva biopsies following our standard operating procedures.
Success rate of CALEC manufacturing: we were able to generate a cellular graft with a success rate of 96.2% (n=132 biopsies, in 17±5 days, from n=37 different donors with an age range 14-77 years old.

### Example 2: Mock Surgical Transplantation of CALEC Graft

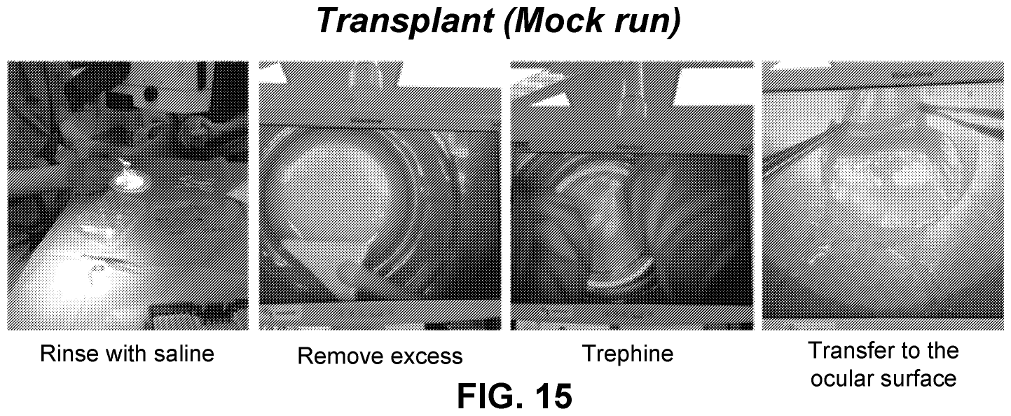

As shown in FIG. 15, the surgical team was able to manipulate and assess CALEC during “mock” transplantation and found no intrinsic difficulty with surgical manipulation of the cellular graft.

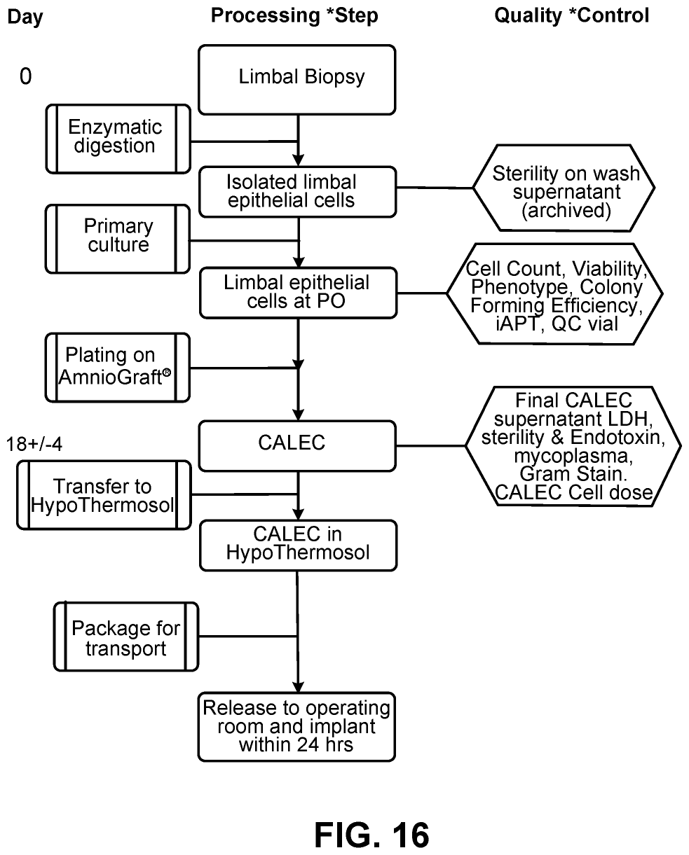

A schematic overview of the FDA-approved clinical manufacturing process that resulted from the process and product development described above is illustrated in FIG. 16.

### Example 3: Transplantation of CALEC Graft

### Preparation (CALEC)

### Limbal Biopsy

Two days prior to biopsy of the donor eye, all participants are started on a topical fluoroquinolone. Some participants are also started on vancomycin in the eye to be biopsied if the participants are Methicillin-resistant Staphylococcus aureus (MRSA) positive or are considered to be part of high-risk populations (e.g., health care personnel).

Transplantation Two days prior to transplantation, the participant is started on a topical fluoroquinolone (all participants) and potentially vancomycin drops (in participants that are MRSA positive or in high-risk populations, e.g., health care personnel) in the recipient eye.

### Administration

### CALEC Procedure—Biopsy

1. Participant is taken to the preoperative area of the ambulatory surgery center where standard operating procedures (SOP) are employed in preparing the donor eye for the surgery.
2. Type of anesthesia is decided depending upon the age and overall functioning of the participant. If the procedure is performed under general anesthesia (GA), GA consent is obtained from the participant or guardian. Otherwise, monitored intravenous anesthesia (MIVA) is performed after the consent.
3. The eye that is to be biopsied is marked as such and the other eye is covered.
4. Fluoroquinolone and proparacaine drops are administered 3 times prior to the procedure.
5. Lidocaine 1% gel is administered into the eye and the eye is closed with tape.
6. The participant is brought to the operating suite and positioned in a supine position under the operating microscope in the manner typical for ophthalmic surgery.
7. The operative eye is cleaned using 5% Betadine solution per standard surgical protocol. Both the cul-de-sac and the eyelashes are cleaned.
8. Under sterile conditions, a limbal biopsy of 3 mm-by-3 mm (1 clock hour) is dissected from superior or inferior portion of the eye, at the discretion of the operating surgeon. The actual size of the graft is measured and captured for data collection.
9. The biopsied material is placed into the container with HypoThermosol® FRS for transfer to the Dana-Farber Cancer Institute.
10. The biopsied site in the conjunctiva is closed using interrupted 8-0 Vicryl sutures.
11. Maxitrol or Tobradex ointment or drops is placed onto the eye.
12. Either a patch or shield is placed over the eye.

### CALEC Procedure—Transplantation

1. The participant is taken to the preoperative area of the ambulatory surgery center where standard operating procedures (SOP) are employed in preparing the recipient eye for the surgery.
2. Type of anesthesia is decided depending upon the age and overall functioning of the participant. If the procedure is performed under general anesthesia (GA), GA consent is obtained from the participant or guardian. Otherwise, monitored intravenous anesthesia (MIVA) is performed after the consent.
3. In cases of MIVA anesthesia, peribulbar block is injected in the operated eye.
4. The recipient eye that is operated on (i.e., the eye with LSCD) is marked as such and the other eye is covered.
5. Fluoroquinolone and proparacaine drops are administered 3 times prior to the procedure.
6. Peribulbar or retrobulbar block with 50:50 mixture of lidocaine and bupivacaine is injected into the recipient eye.
7. The participant is brought to the operating suite and positioned in a supine position under the operating microscope in the manner typical for ophthalmic surgery.
8. The operative eye is cleaned using 5% Betadine solution per standard surgical protocol. Both the cul-de-sac and the eyelashes are cleaned.
9. If excessive ocular surface bleeding is detected, topical epinephrine (1:10,000) is used to constrict the blood vessels and minimize bleeding prior to or during the procedure. A 360-degree conjunctival peritomy is performed per standard surgical procedure. The fibrovascular tissue is dissected from the limbus and the cornea. Hemostasis is achieved by wet-field cautery.
10. The transwell with CALEC graft inside is removed from the original container and is placed on a sterile silicone platform and rinsed with BSS® Sterile Irrigation Solution. The 14-16 mm (depending on the size of the eye) free-held trephine is used to punch the graft. The transwell with the remnants of the graft is lifted off and sent for quality assurance testing. The trephined CALEC graft on the transwell membrane is lifted with the forceps and transferred to the surgical field where the CALEC graft is peeled from the transwell membrane and centered onto the ocular surface with epithelium side up. CALEC is secured with interrupted 8-0 Vicryl sutures and/or fibrin glue. At the end of the procedure, fluorescein will be used to assess epithelial integrity. Lack of fluorescein uptake in the central cornea indicates that epithelium of the CALEC graft is intact.
11. Bandage contact lens are placed over the graft.
12. Subconjunctival injection of Kefzol and Decadron are given.
13. Maxitrol or Tobradex ointment or drops are placed onto the eye.
14. Either a patch or shield is placed over the eye.

### Example 4: Evaluation of the ‘Sternness’ in CALEC Construct

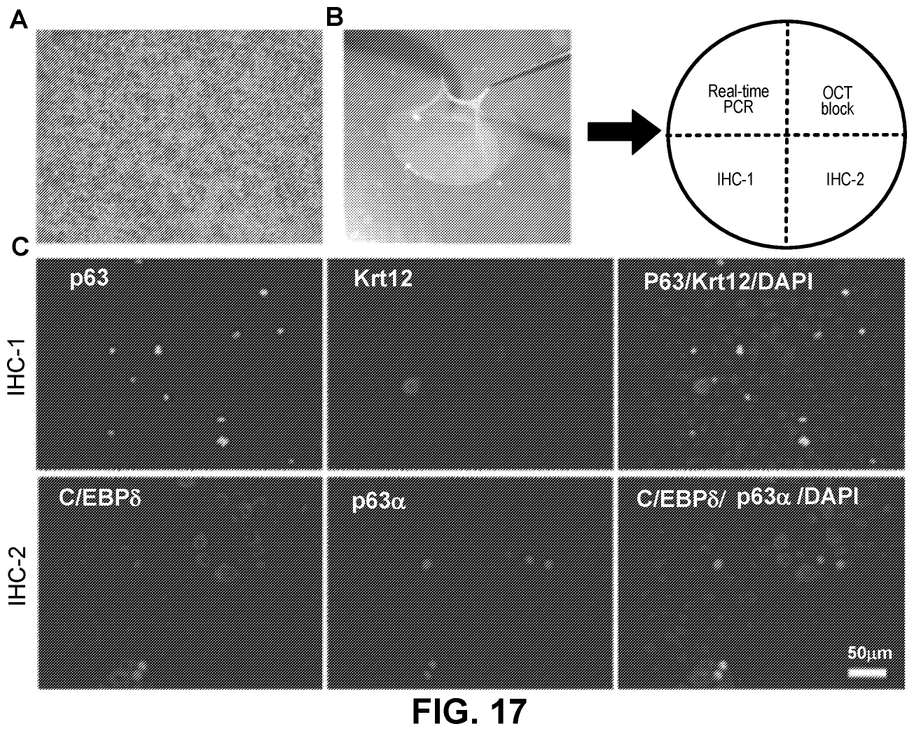

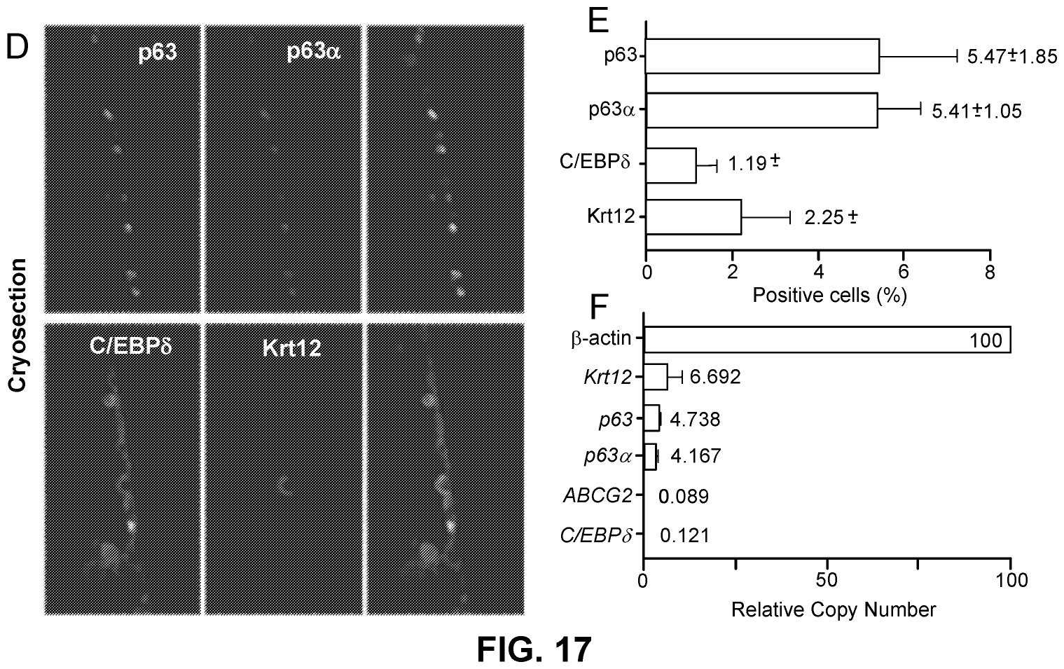

The ‘sternness’ of the CALEC graft was evaluated. FIG. 17A shows the CALEC graft construct. To evaluate the CALEC graft for its ‘sternness’, the graft was divided into 4 quandrants. 1 quandrant was for qRT-PCR analysis, 2 quandrants were for whole-mount immunostaining analysis, and 1 quandrant was embedded in OCT solution for cryo-section in case of technical failure with whole-mount immunostaining. Immonstaining and cryosection was done by standard methods well known in the art.

The cell morphology of CALEC construct is shown in FIG. 17B.

Representative images of p63, p63α, C/EBPδ, Krt12 immunofluorescent staining are shown in whole-mount (FIG. 17C) and Cryo-section (FIG. 17D) method. The rate of p63, p63a, C/EBPδ and Krt12 positive cells was counted after imaging (n=4) and is shown in FIG. 17E. FIG. 17F shows the qRT-PCR test results and the relative copy number of p63, p63a, C/EBPδ, ABCG2 and Krt12 genes in CALEC graft when the copy number of endogenous reference gene (3-actin is 100 (n=7). Results show that ‘sternness’ was maintained in the CALEC graft.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for preparing a cultivated autologous limbal epithelial cell (CALEC) graft for surgical transplantation, the method comprising:
i) providing a tissue from a limbal biopsy or from a wet mucosal source;
ii) treating the tissue from the limbal biopsy or the wet mucosal source with an enzyme blend thereby isolating limbal epithelial cells;
iii) culturing the limbal epithelial cells in the presence of serum-free complete corneal epithelial cell medium for a sufficient period of time until the limbal epithelial cells reach 70-90% confluence;
iv) detaching the limbal epithelial cells;
v) seeding the limbal epithelial cells on a suitable membrane substrate;
vi) growing the cells on the suitable membrane substrate for a sufficient period of time until the limbal epithelial cells reach 70-80% confluence thereby producing a CALEC graft; and
vii) immersing the CALEC graft in a preservation medium suitable for hypothermic bio-preservation until surgical transplantation.

2. A method of treating limbal stem cell deficiency, comprising:
i) obtaining a tissue from a limbal biopsy or from a wet mucosal source;
ii) treating the tissue from the limbal biopsy or the wet mucosal source with an enzyme blend thereby isolating limbal epithelial cells;
iii) culturing the limbal epithelial cells in the presence of serum-free complete corneal epithelial cell medium for a sufficient period of time until the limbal epithelial cells reach 70-90% confluence;
iv) detaching the limbal epithelial cells;
v) seeding the limbal epithelial cells on a suitable membrane substrate;
vi) growing the cells on the suitable membrane substrate for a sufficient period of time until the limbal epithelial cells reach 70-80% confluence thereby producing CALEC graft;
vii) immersing the limbal epithelial cells in a preservation medium suitable for hypothermic bio-preservation until surgical transplantation; and
viii) transplanting the CALEC graft onto an affected eye of the patient.

3. The method according to claim 1 or 2, wherein the medium used in step iii) is free from non-human animal derived products.

4. The method according to any preceding claim, wherein the biopsy is collected in a sterile container filled with the preservation medium suitable for hypothermic bio-preservation.

5. The method according to any preceding claim, wherein the enzyme blend comprises collagenase class I and class II, and an animal-free serine protease.

6. The method according to any preceding claim, further comprising after step iv) washing the limbal epithelial cells with and resuspending the cells in epithelial cell culture medium.

7. The method according to any preceding claim, further comprising prior to step v) de-epithelializing the suitable membrane substrate and seeding the de-epithelialized suitable membrane substrate into a transwell insert in preparation for seeding the limbal epithelial cells.

8. The method according to any preceding claim, further comprising after step vii) maintaining the CALEC graft immersed in the preservation medium at a temperature ranging from 1-10° C.

9. The method according to any preceding claim, wherein step v) comprises seeding 2.5-5×10⁴ limbal epithelial cells onto the suitable membrane substrate.

10. The method according to any preceding claim, wherein step ii) yields 3-7×10⁴ limbal epithelial cells.

11. The method according to any preceding claim, wherein the sufficient period of time in step iii) ranges from 6 to 10 days.

12. The method according to any preceding claim, wherein the sufficient period of time in step vi) ranges from 6 to 10 days.

13. The method according to any preceding claim, wherein step iii) further comprises changing the medium every 2 to 3 days.

14. The method according to any preceding claim, wherein step vi) further comprises changing the medium every 1 to 3 days.

15. The method according to any preceding claim, wherein the limbal biopsy or the wet mucosal source originates from a patient suffering from a limbal stem cell deficiency.

16. The method according to any preceding claim, wherein the limbal biopsy or the wet mucosal source originates from an allogeneic donor.

17. The method according to claim 16, wherein the allogeneic donor is live.

18. The method according to claim 16, wherein the allogeneic donor is cadaveric.

19. The method according to any preceding claim, wherein the wet musosal source is oral mucosa or conjunctiva.

20. The method according to any preceding claim, wherein the suitable membrane substrate is an amniotic membrane.

21. The method according to claim 20, wherein the amniotic membrane is a human amniotic membrane.

22. The method according to any one of claims 1-19, wherein suitable membrane substrate is a basement membrane.

23. The method according to any preceding claim, wherein the limbal epithelial cells are positive for CD49F, CD49E, CD326, CD318, and CD340, and are negative for CD3, CD14, CD16, CD19, CD20, CD56, CD45, CD31.

24. A population of cells produced by steps i) to iii) of claim 1 or claim 2, wherein the cells are positive for CD49F, CD49E, CD326, CD318, and CD340, and are negative for CD3, CD14, CD16, CD19, CD20, CD56, CD45, CD31.

25. A method of producing a population of cells positive for CD49F, CD49E, CD326, CD318, and CD340, and are negative for CD3, CD14, CD16, CD19, CD20, CD56, CD45, CD31, the method comprising:
i) providing a tissue from a limbal biopsy or from a wet mucosal source;
ii) treating the tissue from the limbal biopsy or the wet mucosal source with an enzyme blend thereby isolating limbal epithelial cells; and
iii) culturing the limbal epithelial cells in the presence of serum-free complete corneal epithelial cell medium for a sufficient period of time until the limbal epithelial cells reach 70-90% confluence.
